(19) United States
(12) Patent Application Publication
(10) Pub. No.: US 2026/0337133 A1
(43) Pub. Date: Oct. 29, 2026

(54) PROCESS AND PLANT FOR PRODUCING VINYL CHLORIDE FROM 1,2-DICHLOROETHANE

(71) Applicants: thyssenkrupp Uhde GmbH, Dortmund (DE); thyssenkrupp AG, Essen (DE); Westlake Vinnolit GmbH & Co. KG, Ismaning (DE)

(72) Inventors: Michael BENJE, Bad Soden (DE); Peter KAMMERHOFER, Burgkirchen (DE); Klaus KREJCI, Burghausen (DE)

(73) Assignees: thyssenkrupp Uhde GmbH, Dortmund (DE); thyssenkrupp AG, Essen (DE); Westlake Vinnolit GmbH & Co. KG, Ismaning (DE)

(21) Appl. No.: 19/107,118

(22) PCT Filed: Aug. 21, 2023
(86) PCT No.: PCT/EP2023/072946

§ 371 Date: Feb. 27, 2025

(30) Foreign Application Priority Data
Aug. 29, 2022 (DE) 10 2022 208 894.8
Aug. 29, 2022 (LU) LU102998

(51) Int. Cl. C07C 17/23; B01J 8/06

(52) U.S. Cl. CPC C07C 17/23; B01J 8/065; B01J 8/067; B01J 2208/00176; B01J 2208/00194; B01J 2208/00256; B01J 2208/00504

(57) ABSTRACT

A process for producing vinyl chloride by catalytic thermal cleavage of 1,2-dichloroethane comprises supplying heat required for the thermal cleavage via a liquid heat-transfer medium or condensing heat-transfer medium, wherein the heat-transfer medium is heated at least partly by means of the thermal energy produced in the oxidation of hydrogen, ammonia, or a hydrogen-ammonia mixture. A plant for producing vinyl chloride by catalytic thermal cleavage of 1,2-dichloroethane comprises at least one reactor in which the thermal cleavage takes place and also at least one first heating device by which the reaction medium is heated in the reactor by the heat-transfer medium, the at least one first heating device being designed for an oxidation of hydrogen and/or ammonia, for example a combustion of hydrogen and/or ammonia, whereby thermal energy that can be used to heat the heat-transfer medium is generated.

The present invention relates to a process for producing vinyl chloride by catalytic thermal cleavage of 1,2-dichloroethane, in which the heat required for the thermal cleavage is supplied via a liquid heat-transfer medium or condensing heat-transfer medium. The present invention additionally provides a plant for producing vinyl chloride by catalytic thermal cleavage of 1,2-dichloroethane, in which the heat required for the thermal cleavage is supplied via a liquid heat-transfer medium or condensing heat-transfer medium, said plant including at least one reactor in which the thermal cleavage takes place, at least one first heating device by means of which the heat-transfer medium is heated, and a conduit system for supplying the heated heat-transfer medium (4) to the reactor (1). The at least one first heating device is operated by means of a fuel that burns without the formation of carbon dioxide and is preferably hydrogen or ammonia or a mixture of hydrogen and ammonia, more preferably hydrogen, i.e. the heat-transfer medium is heated in the at least one first heating device by means of thermal energy generated through the oxidation of hydrogen or of another fuel that burns without the formation of carbon dioxide.

The thermal cleavage of 1,2-dichloroethane to produce vinyl chloride, which is required in particular for the production of polyvinyl chloride, follows the reaction equation shown below:

C₂H₄Cl₂→C₂H₄Cl+HCl (1)

This is an endothermic reaction in which the pyrolysis can either be carried out without catalyst in the gas phase under high pressure of 1 to 3 MPa and at a temperature of 450 to 600 °C, or in catalytic processes that allow the pyrolysis to be carried out at lower temperatures.

### PRIOR ART

EP 0 264 065 A1, for example, describes a process for producing vinyl chloride by catalyst-free thermal cleavage of 1,2-dichloroethane, in which 1,2-dichloroethane is heated in a first container, then transferred to a second container by vaporizing it without further heating under lower pressure than in the first container and feeding the gaseous 1,2-dichloroethane into a cracking furnace in which the cleavage into vinyl chloride and hydrogen chloride takes place.

The temperature of the 1,2-dichloroethane on exiting the second container is 220 °C to 280 °C. In the cracking furnace, tubes in which the 1,2-dichloroethane undergoes thermal cleavage are heated by means of a fossil fuel. In the radiation zone of the cracking furnace the gaseous 1,2-dichloroethane is heated to 525 °C and 533 °C respectively.

EP 0 264 065 A1 also mentions that for preheating the liquid, fresh 1,2-dichloroethane it is possible to use a temperature-regulating medium that is in turn heated in the convection zone of the cracking furnace by the flue gas generated by the burners heating the cracking furnace.

Heated high-boiling liquids such as mineral oil, silicone oil or molten biphenyl are suitable as the temperature-regulating medium. However, this achieves only preheating to a temperature of 150 to 220 °C, whereas the pyrolysis itself is carried out at temperatures of about 530 °C. This known process therefore does not provide for the performance of the pyrolysis at temperatures in the range from 200 to 400 °C and for all the heating with the aid of a liquid heat-transfer medium that is necessary here.

A plant complex for producing vinyl chloride generally consists of
a plant for producing 1,2-dichloroethane from ethene and chlorine (“direct chlorination”), or
a plant for producing 1,2-dichloroethane from ethene, hydrogen chloride and oxygen (“oxychlorination”),
a plant for the distillative purification of 1,2-dichloroethane,
a plant for the thermal cleavage of the distillatively purified 1,2-dichloroethane to vinyl chloride and hydrogen chloride and
a plant for the distillative removal of hydrogen chloride and unreacted 1,2-dichloroethane and for purification of the vinyl chloride.

The hydrogen chloride obtained by thermal cleavage of the 1,2-dichloroethane can be returned to the oxychlorination plant, where it can be reacted again with ethene and oxygen to form 1,2-dichloroethane.

The plant complex described above may additionally include a plant for incinerating liquid and/or gaseous chlorinated hydrocarbons. These are produced as by-products in the vinyl chloride production process and are removed primarily in the distillative purification of 1,2-dichloroethane. The hydrogen chloride formed in the incineration of these substances is either supplied as aqueous hydrochloric acid to other production processes or is likewise returned to the oxychlorination plant. The waste heat from the incineration is used in existing steam-generation processes.

In the process described in DE 102 52 891 A1 for the cleavage of 1,2-dichloroethane into vinyl chloride and hydrogen chloride, a catalyst is used that makes it possible to lower the operating temperature in the endothermic cleavage. However, in this process too, the tubular reactor is fired by a primary energy carrier such as oil or gas, the furnace being divided into a radiation zone and a convection zone. In the radiation zone, the heat required for the pyrolysis is transferred to the reaction tube primarily by radiation from the burner-heated furnace walls. In the convection zone, the energy content of the hot flue gases exiting the radiation zone is utilized through convective heat transfer, as a result of which the 1,2-dichloroethane can be preheated, vaporized or superheated as the reactant in the pyrolysis reaction.

Various measures for energy saving and/or heat recovery in plants for producing 1,2-dichloroethane are known from the prior art. Such measures result in a significant lowering in operating costs and thus contribute significantly to the economic efficiency of the plant and to reducing the CO₂ emissions of the plant. These are for example measures that utilize the heat of reaction of the exothermic reaction steps for the heating of heat sinks in the process. WO 2014/108159 A1 details various known measures for heat recovery in plants for producing vinyl chloride, citing the corresponding references.

EP 0 225 617 A1 describes a process for producing vinyl chloride by thermal cleavage of 1,2-dichloroethane, it being mentioned therein that the performance of this process provides in some cases for the recovery of waste heat from the flue gases of a fired cracking furnace through the generation of steam. However, the relatively low flue gas temperature means that such processes are not very economical. The thermal cleavage of 1,2-dichloroethane takes place at comparatively high temperatures in this process too. First, the reactant is preheated to about 243 °C, then vaporized partly through pressure release and partly by pressurizing with steam, and then thermally cleaved in a cracking furnace at temperatures of between 435 °C and 497 °C without the use of a catalyst. Heating by means of a heat-transfer oil is not described and is also not possible at these temperatures.

EP 0 002 021 A1 describes a process for the catalytic dehydrohalogenation of 1,2-dichloroethane to vinyl chloride using zeolite catalysts treated with a Lewis acid. When using such catalysts, the reaction can be carried out at elevated pressure and temperatures in the range of from 200 °C to 400 °C and thus at significantly lower temperatures than in the conventional pyrolysis of 1,2-dichloroethane.

The object of the present invention is to provide an improved process for producing vinyl chloride by thermal cleavage of 1,2-dichloroethane in which a reduction in operating costs is achieved. A further object of the present invention is to improve the CO₂ balance of a production process for producing vinyl chloride.

The above object is achieved by a process for producing vinyl chloride by catalytic thermal cleavage of 1,2-dichloroethane of the nature mentioned in the introduction and having the features of claim 1.

According to the invention, the liquid or condensing heat-transfer medium is heated at least temporarily at least partly by means of thermal energy produced in the oxidation of hydrogen, ammonia or an ammonia-hydrogen mixture. Such a mode of operation is CO₂-neutral. The use of catalysts for the thermal cleavage of 1,2-dichloroethane makes it possible to shift the temperature range in which the reaction takes place sufficiently to lower temperatures, more particularly to the range from about 200 °C to about 400 °C, such that the reactor can be heated by means of a heat-transfer medium instead of the direct firing with fossil fuels employed in existing processes. Instead of a tubular cracking furnace it is possible to employ as the reactor for example a shell-and-tube heat exchanger in which the tubes are packed with a catalyst bed and the heat-transfer medium flows through the shell space, preferably in a loop.

According to a preferred development of the process of the invention, at least some or all of the hydrogen to be oxidized is produced in an upstream electrolysis unit. The electrolysis unit can preferably be set up for performance of a chlorine-alkali electrolysis. The chlorine gas that is also produced can be used for the purposes of direct chlorination in the production process for vinyl chloride. Alternatively, the electrolysis unit can also be set up for water electrolysis. In this case, the oxygen produced can be used in the oxychlorination. Lastly, the electrolysis unit can also be set up for hydrogen chloride electrolysis. Preferably, the hydrogen chloride produced in the thermal cleavage of 1,2-dichloroethane undergoes electrochemical cleavage and/or the chlorine gas that is formed is reused for the chlorination of ethene in the production process.

In addition, it is advantageous for the heat required for the reaction to be obtained at least temporarily partly through heating the heat-transfer medium by combustion of hydrogen and/or ammonia. It is possible here for the heat needed for the reaction to be obtained entirely through the oxidation of hydrogen and/or ammonia.

It is also possible for the heat-transfer medium to be heated at least temporarily partly by means of thermal energy produced through oxidation of hydrogen and/or ammonia and partly by combustion of a fuel different from these fuels. With this preferred variant of the process, the heat required for the reaction is mostly provided via a first heating device heatable by means of hydrogen and/or ammonia, but at least one second heating device operated by means of a fuel different from the above fuels is provided and can be utilized at least temporarily. In these cases, the first heating device can be throttled or, if necessary, shut down completely for a certain period of time, or the heat-transfer medium conducted in such a way that its flow bypasses the first heating device.

For example, the at least one second heating device may be designed as an incineration plant for incinerating liquid and/or gaseous chlorinated hydrocarbons such as those produced as by-products in a vinyl chloride production plant.

The use of a liquid or condensing heat-transfer medium for providing the totality of the reaction heat needed for the pyrolytic cleavage of the 1,2-dichloroethane is made possible by the performance of the reaction in the presence of suitable catalysts that allow a substantial lowering of the reaction temperature compared to conventional processes without catalysis.

When such catalysts are used, the reaction temperature can be lowered for example from the temperatures of the order of about 430 °C to about 530 °C that are usual in conventional processes to temperatures in the range of in particular about 200 °C to 400 °C. Heating to temperatures in this range is possible for example when using a heat-transfer oil or optionally a molten salt. Suitable catalysts include for example substances such as those named in EP 0 002 021 A1 mentioned above.

A process for purely thermal (uncatalysed in a pyrolysis furnace) or thermal-catalytic EDC cleavage (with the supply of heat and use of a catalyst) normally consists of the substeps of:
preheating liquid 1,2-dichloroethane up to the vaporization temperature at the given pressure,
vaporizing the preheated 1,2-dichloroethane,
optionally superheating the 1,2-dichloroethane vapour up to the range of the reaction temperature (if the prior vaporization did not take place in the range of the reaction temperature),
performing the cleavage reaction (purely thermally, or thermally with the use of a catalyst) with the supply of heat.

The invention provides a process which, in addition to the heating for the catalytic-thermal cleavage reaction by means of a liquid or condensing heat-transfer medium, also enables the heating for the upstream preheating, vaporization or superheating of the 1,2-dichloroethane by said heat-transfer medium. It is not absolutely necessary for all these steps to be heated by means of the heat-transfer medium. The process of the invention comprises the heating of at least one up to any desired combination of the above substeps, it being possible in turn for the individual substeps to be subdivided (in terms of apparatus) into individual steps. “Heating” for the purposes of the process of the invention means the transfer of heat to the starting material 1,2-dichloroethane and/or to the reaction mixture by a heat-transfer medium.

This can result in the starting material 1,2-dichloroethane being heated, vaporized and/or superheated. Heat can be supplied to the reaction mixture in the reactor at a constant temperature level (isothermal reaction control). The reaction mixture can also be heated further, with the heat supplied by the heating used in part to cover the heat requirement for the reaction and in part for the further heating of the reaction mixture. Lastly, the supply of heat to the reaction mixture by the heating can be adjusted such that the sensible heat content of the reaction mixture is used at least in part to cover the heat requirement for the reaction and the reaction mixture in the reactor cools down by comparison with the reactor inlet temperature. The heating and also the transfer of heat to the starting material 1,2-dichloroethane is effected by means of by a liquid heat-transfer medium accompanied by cooling of the heat-transfer medium/a reduction in the sensible heat content thereof and/or by means of a condensing heat-transfer medium that had previously been vaporized by means of a heating device.

Heating devices for the heat-transfer medium may for the purposes of the process of the invention be devices (heaters and/or evaporators or devices combining the functions of a heater and an evaporator) that can be heated by means of hydrogen, ammonia or an ammonia-hydrogen mixture (first heating device) or a fossil fuel such as heating oil or preferably natural gas (second heating device). Alternatively, they may be heat-transfer devices (heaters and/or evaporators or devices combining the functions of a heater and an evaporator) heated by means of waste heat from a plant for incinerating by-products of a chemicals plant, preferably from a plant for incinerating by-products of a plant complex for vinyl chloride production. Such devices are known to those skilled in the art.

As a rule, the heat requirement of a plant for the catalytic-thermal cleavage of 1,2-dichloroethane can be met only partially by the incineration of by-products of the plant complex for vinyl chloride production. Thus, in a preferred mode of operation, the heat-transfer medium is initially heated by means of the waste heat from the incineration of by-products and the remainder of the heat required is supplied by combustion of hydrogen, ammonia or a hydrogen-ammonia mixture in the first heating unit.

According to a preferred development of the process of the invention, the liquid or condensing heat-transfer medium is circulated in a loop, said loop including the at least one first heating device operated through oxidation of hydrogen, ammonia or a hydrogen-ammonia mixture and a reactor for the catalytic thermal cleavage of 1,2-dichloroethane, wherein an exchange of heat takes place between a reaction medium in the reactor and the heat-transfer medium. The loop described above preferably also includes at least one second heating device operated through combustion of at least one fuel different from the fuels mentioned above.

According to a preferred development of the process of the invention, at least one first heating device and at least one second heating device are connected in series in the loop. In this case, the heat-transfer medium flows in a conduit loop first through the second heating device and then through the first heating device situated downstream thereof; alternatively, the flow through these two heating devices can take place the other way round. Alternatively, it is also possible for the two heating devices to be arranged in a quasi-parallel connection, i.e. the connections of the conduit loop that includes the heating devices are such that the relevant conduits can be closed off, for example via valves, so that the heat-transfer medium is able to flow through the second heating device without also flowing through the first heating device, or vice versa as necessary.

According to a preferred development of the process of the invention, the heat-transfer medium is circulated in the loop in counterflow to the flow of the reaction medium through the reactor.

This variant is advantageous for effective heat transfer. Alternatively, a flow of the heat-transfer medium in cocurrent flow to the flow of the reaction medium is possible.

According to a preferred development of the process of the invention, the second heating device is operated at least temporarily by means of the waste heat from a plant for incinerating the by-products of a vinyl chloride production plant. This variant has the advantage that the waste heat used comes essentially from a plant element within the same plant complex, which means that the energy balance of the process can be improved.

According to a preferred development of the process of the invention, the second heating device is operated permanently at full load. The remainder of the energy required for thermal cleavage can be supplied to the heat-transfer medium by means of a first heating unit heated by hydrogen and/or ammonia. In this variant of the process, the second heating device is preferably permanently at operating temperature.

According to a preferred development of the process of the invention, the thermal cleavage of the 1,2-dichloroethane is carried out within a temperature range of from 200 °C to 400 °C. This is a preferred temperature range that can be easily achieved with liquid heat-transfer media, for example heat-transfer oils.

The invention further provides a plant for producing vinyl chloride by catalytic thermal cleavage of 1,2-dichloroethane, in which the heat required for the thermal cleavage of the 1,2-dichloroethane and preferably also for the preheating, vaporization and/or superheating of the 1,2-dichloroethane is supplied via a liquid heat-transfer medium or condensing heat-transfer medium, said plant including at least one reactor in which the thermal cleavage takes place, at least one first heating device by means of which the liquid heat-transfer medium is heated and a conduit system for supplying the heated heat-transfer medium to the reactor, wherein the at least one first heating device is designed for the oxidation of hydrogen and/or ammonia, i.e. it is able to generate heat through the oxidation of one of the abovementioned fuels, preferably by combustion.

It is particularly preferable here when the plant includes at least one electrolysis unit upstream of the at least one first heating device, especially for the performance of a chlorine-alkali electrolysis, a water electrolysis or a hydrogen chloride electrolysis, wherein the hydrogen produced-optionally also after admixing ammonia-is supplied to the at least one first heating device.

In addition, the plant may include at least one second heating device for heating the reaction medium that is designed for the combustion of at least one fuel different from hydrogen and ammonia, for example methane, natural gas and/or liquid and/or gaseous waste from a chemicals plant. In this case, it is preferable that the heat-transfer medium flows through the second heating device first. The remainder of the heat required for the cleavage of the 1,2-dichloroethane and also for its preheating, in order to vaporize and/or superheat it, can be supplied through the first heating unit. The first heating unit can preferably be designed to be regulatable for reaching a predeterminable final temperature in the heat-transfer medium.

In a preferred development of the invention, the conduit system forms a loop of the heat-transfer medium that includes the reactor and the at least one first heating device operated by means of hydrogen, ammonia or a hydrogen-ammonia mixture.

According to a preferred variant of the invention, the loop of the heat-transfer medium also includes the at least one second heating device. In this case, the order of flow is preferably such that the medium flows through the second heating device first. The terms “first” or “second” heating device referred to here thus refer merely to the functionally different nature of the heating devices and do not define the order in which the heat-transfer medium flows through them.

In a preferred development of the invention, the first heating device and the second heating device are arranged in the loop of the heat-transfer medium in series or alternatively in parallel. According to a preferred variant of the invention, means are provided for transferring heat from the heat-transfer medium to a reaction medium flowing through the reactor or present in the reactor.

In a preferred development of the invention, the reactor comprises a shell-and-tube heat exchanger, the tubes of which are packed with a catalyst bed and which preferably has a shell space through which the heat-transfer medium circulating in the loop flows.

The present invention will be described in more detail below on the basis of an exemplary embodiment with reference to the appended drawings in which:

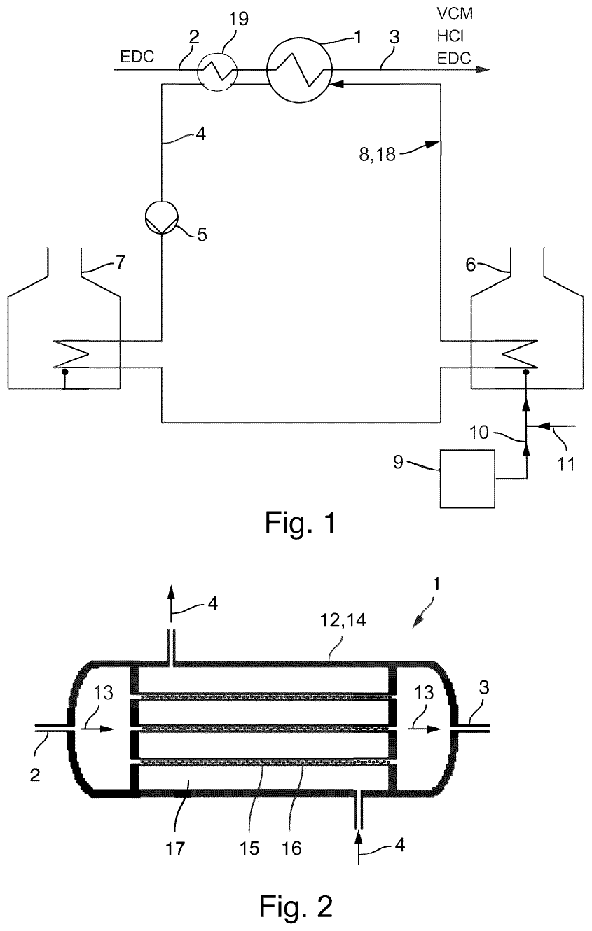

FIG. 1 shows a schematically simplified plant diagram of a plant according to the invention for producing vinyl chloride by catalytic thermal cleavage of 1,2-dichloroethane, and

FIG. 2 shows a schematic representation of a reactor comprising a shell-and-tube heat exchanger, the tubes of which are packed with a catalyst bed.

Reference is made below to the figures, which are used to elucidate an exemplary embodiment variant of the process of the invention in more detail. The depictions in FIGS. 1 and 2 are schematically greatly simplified and only those plant components that are relevant in the context of the present invention are shown. The plant comprises a reactor 1, to which, for example via at least one conduit, a reactor inlet stream 2 of 1,2-dichloroethane (EDC) is supplied, which is pyrolysed into monomeric vinyl chloride (VCM) in reactor 1 under the influence of heat, the formation of vinyl chloride being accompanied by the evolution of hydrogen chloride. The recited products of the process exit reactor 1 in a reactor discharge stream 3.

The reactor 1 is integrated into a loop 18 of a heat-transfer medium 4 formed by a conduit system 8 such that heat is supplied to the reactor 1 via the liquid heat-transfer medium, for example a heat-transfer oil, which preferably flows in counterflow to the reaction medium, so as to heat the reaction medium 13 flowing through the reactor 1 (see FIG. 2) to a temperature of, for example, 300 °C to 400 °C, at which the catalytic thermal cleavage of 1,2-dichloroethane to vinyl chloride takes place in reactor 1.

The loop 18 of the heat-transfer medium 4 is elucidated in more detail below. The loop 18 of the heat-transfer medium 4 includes a pump 5 for circulating around the heat-transfer medium 4 in the loop 18, wherein downstream of the pump 5 the heat-transfer medium first flows through a second heating device 7 that is according to the teaching of the invention optional, in which the heat-transfer medium 4, if the second heating device 7 is in operation, can be heated by combustion of at least one fuel different from hydrogen and/or ammonia, for example methane, natural gas and/or liquid and/or gaseous waste from a chemicals plant or by heat energy from the waste heat of a chemicals plant, for example from a vinyl chloride production plant.

Downstream of the second heating device 7, the heat-transfer medium 4 flows through a first heating device 6 that is fired with hydrogen 10, ammonia 11 or an ammonia-hydrogen mixture, so as to heat the heat-transfer medium 4. As shown in FIG. 1, the hydrogen 10 used for heating can be produced for example in an upstream electrolysis unit 9. The electrolysis unit 9 can preferably be set up for performance of a chlorine-alkali electrolysis, a water electrolysis or else a hydrogen chloride electrolysis.

In the exemplary embodiment, the second heating device 7 and the first heating device 6 are in the conduit system 8 of the loop 18 arranged one after the other in the direction of flow and are thus connected in series. Alternatively, the two heating devices can however also be connected in parallel to one other, i.e. unlike in FIG. 1 the two heating devices are integrated into a conduit system 8 such that the heat-transfer medium 4 is able at least also to flow through one or the other of the two heating devices while bypassing the other heating device.

In the variant shown in FIG. 1 in which the two heating devices 6 and 7 are arranged in series and also in the variant with parallel connection that is not shown, valves not illustrated in FIG. 1 may be provided to switch the heating devices on and off or to shut off the conduits in the loop 18 at appropriate places. In addition, one or more regulation devices may be provided (also not shown in FIG. 1) so as to regulate the heat output supplied by the first and/or second heating device 6 and 7 in accordance with the heating requirement of the reaction medium in the reactor 1.

A variant of the invention shown in FIG. 1 optionally additionally includes a device 19 in which the reactor inlet stream 2 can be preheated, vaporized and superheated by means of the heat content of the flow of the heat-transfer medium 4, wherein these options do not necessarily all have to be realized, but can be realized in any combination. The device 19 is arranged in the loop of the heat-transfer medium preferably downstream of the reactor 1.

### LIST OF REFERENCE NUMERALS

1 Reactor
2 Reactor inflow stream
3 Reactor outflow stream
4 Heat-transfer medium
5 Recirculation pump
6 First heating device
7 Second heating device
8 Conduit system
9 Electrolysis unit
10 Hydrogen
11 Ammonia
12 Heat-transfer means
13 Reaction medium
14 Shell-and-tube heat exchanger
15 Tubes
16 Catalyst bed
17 Shell space
18 Loop
19 Device for preheating and/or vaporization and/or superheating

1-21. (canceled)

22. A process for producing vinyl chloride by catalytic thermal cleavage of 1,2-dichloroethane, comprising:
supplying heat required for the thermal cleavage via a liquid heat-transfer medium or condensing heat-transfer medium, wherein the heat-transfer medium is heated at least temporarily at least partly by thermal energy produced in the oxidation of hydrogen, ammonia or a hydrogen-ammonia mixture.

23. The process according to claim 22, wherein at least some or all of the hydrogen to be oxidized is produced in an upstream electrolysis unit.

24. The process according to claim 22, wherein the oxidation of the hydrogen and/or of the ammonia is carried out by combustion of the hydrogen and/or of the ammonia.

25. The process according to claim 22, wherein the 1,2-dichloroethane is preheated and/or vaporized and/or superheated by the heat-transfer medium.

26. The process according to claim 22, wherein the heat-transfer medium is heated at least temporarily partly by thermal energy produced through oxidation of hydrogen and/or ammonia and partly by combustion of a fuel different from hydrogen and ammonia, or the heat-transfer medium is heated entirely by thermal energy produced in the oxidation of hydrogen and/or ammonia.

27. The process according to claim 22, wherein at least one first heating device operated by oxidation of hydrogen and/or ammonia and additionally at least one second heating device operated by combustion of at least one fuel different from hydrogen and ammonia are used for heating the liquid heat-transfer medium.

28. The process according to claim 22, wherein the heat-transfer medium is circulated in a loop and said loop includes the at least one first heating device and a reactor for the catalytic thermal cleavage of 1,2-dichloroethane, wherein an exchange of heat takes place between a reaction medium in the reactor and the heat-transfer medium.

29. The process according to claim 22, wherein:
at least one first heating device operated by oxidation of hydrogen and/or ammonia and additionally at least one second heating device operated by combustion of at least one fuel different from hydrogen and ammonia are used for heating the liquid heat-transfer medium;
the heat-transfer medium is circulated in a loop and said loop includes the at least one first heating device and a reactor for the catalytic thermal cleavage of 1,2-dichloroethane, wherein an exchange of heat takes place between a reaction medium in the reactor and the heat-transfer medium; and
the at least one first heating device and the at least one second heating device are connected in series in the loop.

30. The process according to claim 29, wherein the heat-transfer medium is circulated in the loop in counterflow to the flow of the reaction medium through the reactor.

31. The process according to claim 22, wherein the thermal cleavage of the 1,2-dichloroethane is carried out within a temperature range of from 200 °C to 400 °C.

32. A plant for producing vinyl chloride by catalytic thermal cleavage of 1,2-dichloroethane, in which heat required for the thermal cleavage of the 1,2-dichloroethane is supplied via a liquid heat-transfer medium or condensing heat-transfer medium, said plant comprising:
at least one reactor in which the thermal cleavage takes place;
at least one first heating device by which the heat-transfer medium is heated; and
a conduit system for supplying the heated heat-transfer medium to the reactor;
wherein the at least one first heating device is configured for oxidation of hydrogen and/or ammonia.

33. The plant according to claim 32, wherein the at least one first heating device is configured for combustion of hydrogen and/or ammonia.

34. The plant according to claim 32, wherein the plant includes at least one electrolysis unit upstream of the at least one first heating device, wherein hydrogen produced in the electrolysis unit is supplied to the at least one first heating device.

35. The plant according to claim 32, wherein the plant additionally includes at least one second heating device for the combustion of at least one fuel different from hydrogen and ammonia for heating the heat-transfer medium.

36. The plant according to claim 32, wherein the conduit system circulates the heat-transfer medium in a loop that includes the reactor and the at least one first heating device.

37. The plant according to claim 32, wherein:
the plant additionally includes at least one second heating device for the combustion of at least one fuel different from hydrogen and ammonia, for example methane, natural gas and/or liquid and/or gaseous waste from a chemicals plant, for heating the heat-transfer medium;
the conduit system circulates the heat-transfer medium in a loop that includes the reactor and the at least one first heating device; and
the loop includes the at least one second heating device.

38. The plant according to claim 37, wherein the at least one first heating device and the at least one second heating device are arranged in the loop of the heat-transfer medium in series or in parallel.

39. The plant according to claim 32, wherein means are provided for transferring heat from the heat-transfer medium to a reaction medium flowing through the reactor.

40. The plant according to claim 32, wherein the reactor comprises a shell-and-tube heat exchanger, the tubes of which contain a flow-through catalyst bed, the heat-transfer medium flowing through a shell space of the reactor.

41. The plant according to claim 32, wherein at least one device for preheating and/or vaporizing and/or superheating the 1,2-dichloroethane is integrated into the conduit system of the heat-transfer medium.
